(19) United States
(12) Patent Application Publication
(10) Pub. No.: US 2015/0305899 A1
(43) Pub. Date: Oct. 29, 2015

(54) BRANCHED POLYHYDROXYALKANOATE SYSTEMS FOR BIORESORBABLE VASCULAR SCAFFOLD APPLICATIONS

(71) Applicant: Abbott Cardiovascular Systems Inc., Santa Clara, CA (US)

(72) Inventors: Joel E. Harrington, Redwood City, CA (US); Mary Beth Kossuth, San Jose, CA (US); James Oberhauser, Saratoga, CA (US)

(73) Assignee: Abbott Cardiovascular Systems Inc., Santa Clara, CA (US)

(21) Appl. No.: 14/261,358

(22) Filed: Apr. 24, 2014

(51) Int. Cl. A61F 2/856

(52) U.S. Cl. CPC A61F 2/856

(57) ABSTRACT

An implantable medical devices such as a stent that includes sparse comb polyhydroxyalkanoate (PHA) systems is disclosed. The stent includes a stent body, scaffold, or substrate made partially or completely of polymer material including PHA.

### BACKGROUND OF THE INVENTION

1. Field of the Invention

This invention relates polymeric medical devices, in particular, bioresorbable stents or stent scaffoldings

2. Description of the State of the Art

This invention relates to radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffolding gets its name because it possibly physically holds open and, if desired, expands the wall of the passageway. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty. Yet, restenosis remains a significant problem. When restenosis does occur in the stented segment, its treatment can be challenging, as clinical options are more limited than for those lesions that were treated solely with a balloon.

Stents are used not only for mechanical intervention but also as vehicles for providing biological therapy. Biological therapy uses medicated stents to locally administer a therapeutic substance. A medicated stent may be fabricated by coating the surface of either a metallic or polymeric scaffold with a polymeric carrier that includes an active or bioactive agent or drug. Polymeric scaffolds may also serve as a carrier of an active agent or drug. An active agent or drug may also be included on a scaffold without being incorporated into a polymeric carrier.

Stents are generally made to withstand the structural loads, namely radial compressive forces, imposed on the scaffold as it supports the walls of a vessel. Therefore, a stent must possess adequate radial strength if its function is to support a vessel at an increased diameter. Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading or pressure, which if exceeded, creates a yield stress condition resulting in the stent diameter not returning to its unloaded diameter, i.e., there is irrecoverable deformation of the stent. See, T. W. Duerig et al., Min Invas Ther & Allied Technol 2000: 9(3/4) 235-246. Stiffness is a measure of the elastic response of a device to an applied load and thus will reflect the effectiveness of the stent in resisting diameter loss due to vessel recoil and other mechanical events. Radial stiffness can be defined for a tubular device such as stent as the hoop force per unit length (of the device) required to elastically change its diameter. The inverse or reciprocal of radial stiffness may be referred to as the compliance. See, T. W. Duerig et al., Min Invas Ther & Allied Technol 2000: 9(3/4) 235-246.

When the radial yield strength is exceeded, the stent is expected to yield more severely and only a minimal force is required to cause major deformation. Radial strength is measured either by applying a compressive load to a stent between flat plates or by applying an inwardly-directed radial load to the stent.

Once expanded, the stent must adequately maintain its size and shape throughout its service life despite the various forces that may come to bear on it, including the cyclic loading induced by the beating heart. For example, a radially directed force may tend to cause a stent to recoil inward. In addition, the stent must possess sufficient flexibility to allow for crimping, expansion, and cyclic loading.

Some treatments with stents require its presence for only a limited period of time. Once treatment is complete, which may include structural tissue support and/or drug delivery, it may be desirable for the stent to be removed or disappear from the treatment location. One way of having a stent disappear may be by fabricating a stent in whole or in part from materials that erode or disintegrate through exposure to conditions within the body. Stents fabricated from biodegradable, bioabsorbable, bioresorbable, and/or bioerodable materials such as bioabsorbable polymers can be designed to completely erode only after the clinical need for them has ended.

In addition to high radial strength, a vascular scaffold must have sufficient resistance to fracture or sufficient toughness. A vascular scaffold is subjected to a large deformation during use, in particular, when it is crimped to a delivery diameter and when it is deployed. A scaffold may be susceptible to fracture when in use which can negatively impact performance and even lead to device failure. Fabricating a polymer-based scaffold that has sufficiently high radial strength as well as resistance to fracture is a challenge.

Additionally, treating peripheral vascular disease percutaneously in the lower limbs is a challenge with current technologies. Long term results are sub-optimal due to chronic injury caused by the constant motions of the vessel and the implant as part of every day life situations. To reduce the chronic injury, a bioresorbable scaffold for the superficial femoral artery (SFA) and/or the popliteal artery can be used so that the scaffold disappears before it causes any significant long term damage. However, one of the challenges with the development of a femoral scaffold and especially a longer length scaffold (4-25 cm) to be exposed to the distal femoral artery and potentially the popliteal artery is the presence of fatigue motions that may lead to chronic recoil and strut fractures especially in the superficial femoral artery, prior to the intended bioresorption time especially when implanted in the superficial femoral artery.

Fabricating a polymer-based scaffold for treating the SFA is even more challenging than for coronary applications. A scaffold in the SFA and/or the popliteal artery is subjected to various non-pulsatile forces, such as radial compression, torsion, flexion, and axial extension and compression. These forces place a high demand on the scaffold mechanical performance and can make the scaffold more susceptible to fracture than less demanding anatomies. Stents or scaffolds for peripheral vessels such as the SFA, require a high degree of crush recovery. The term “crush recovery” is used to describe how the scaffold recovers from a pinch or crush load, while the term “crush resistance” is used to describe the force required to cause a permanent deformation of a scaffold. It has been believed that a requirement of a stent for SFA treatment is a radial strength high enough to maintain a vessel at an expanded diameter. A stent which combines such high radial strength, high crush recovery, and high resistance to fracture is a great challenge.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if each said individual publication, patent, or patent application was fully set forth, including any figures, herein.

### SUMMARY OF THE INVENTION

A first set of embodiments of the present invention includes a stent comprising: a scaffold comprising a branched polyhydroxyalkanoate (PHA) polymer that is a random copolymer having (R)-3-hydroxybutyrate (3HB) units and (R)-3-hydroxyalkanoate (3HA) units with a structure of [(R)-3HB]ₓ—[(R)-3HA]₁₋ₓ, where x is the mol % of (R)-3HB units, wherein the 3HA units have side groups of at least three carbon atoms, wherein the scaffold is radially expandable in a blood vessel of a patient.

The first set of embodiments may have one or more, or any combination of the following aspects (1) to (7): (1) wherein x is between 2% and 50%, (2) wherein the number average molecular weight (Mn) of the PHA polymer is greater than 50 kDa, and (3) wherein the 3HA units are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd), (4) wherein x is between 2% and 15%, (5) wherein the number of side groups is 3 to 7, (6) wherein a crystallinity of the polymer is 20% to 50%, and (7) wherein a Young's modulus of the polymer is greater than 500 MPa and a flexural modulus is 6 to 10 GPa.

A second set of embodiments of the present invention includes a stent comprising: a scaffold comprising a blend of a polylactide—(PLA) based polymer and a branched polyhydroxyalkanoate (PHA) homopolymer, the PHA homopolymer including (R)-3-hydroxybutyrate (3HB) units and (R)-3-hydroxyalkanoate (3HA) units having a structure of [(R)-3HB]ₓ—[(R)-3HA]₁₋ₓ, where x is the mol % of (R)-3HB units, wherein the 3HA units have side groups of at least three carbon atoms, wherein the scaffold is radially expandable in a blood vessel of a patient.

The second set of embodiments may have one or more, or any combination of the following aspects (1) to (10): (1) wherein x is between 2% and 50%, (2) wherein the number average molecular weight (Mn) of the PHA polymer is greater than 50 kDa, (3) wherein the number average molecular weight (Mn) of the PLA-based polymer is greater than 50 kDa, (4) wherein the 3HA units are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd), wherein the PLA-based polymer comprises to 15mol % of D-lactide units, (5) wherein a wt % y of the PHA polymer is 5 to 30 wt % of the blend, (6) wherein a wt % y of the PHA polymer is 70 to 95 wt % of the blend, (7) wherein x is between 2% and 15%, (8) wherein the number of side groups is 3 to 7, (9) wherein a crystallinity of the blend is 20% to 50%, and (10) wherein a Young's modulus of the blend is greater than 500 MPa and a flexural modulus of the blend is 6 to 10 GPa.

A third set of embodiments of the present invention includes a stent comprising: a scaffold comprising a copolymer of a polylactide—(PLA) based polymer and a branched polyhydroxyalkanoate (PHA) polymer, the PHA polymer including (R)-3-hydroxybutyrate (3HB) units and (R)-3-hydroxyalkanoate (3HA) units having a structure of [(R)-3HB]_{xc}—[(R)-3HA]_{1-xc}, where x_{c} is the mol % of (R)-3HB units, wherein the 3HA units have side groups including at least three carbon atoms, wherein the scaffold is radially expandable in a blood vessel of a patient.

The third set of embodiments may have one or more, or any combination of the following aspects (1) to (10): (1) wherein x_{c} is between 2% and 50%, (2) wherein the number average molecular weight (Mn) of the PHA/PLA copolymer is greater than 50 kDa, (3) wherein the 3HA units are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd), (4) wherein the PLA-based polymer comprises 1 to 15mol % of D-lactide units, (5) wherein a mol % y_{c} of the PHA is 5 to 30 mol % of the PHA/PLA copolymer, (6) wherein a mol % y_{c} of the PHA is 70 to 95 mol % of the PHA/PLA copolymer, (7) wherein x is between 2% and 15%, (8) wherein the number of side groups is 3 to 7, (9) wherein a crystallinity of the copolymer is 20% to 50%, and (10) wherein a Young's modulus of the copolymer is greater than 500 MPa and a flexural modulus is 6 to 10 GPa.

A fourth set of embodiments of the present invention includes a stent comprising: a scaffold comprising a blend of a copolymer of a branched PHA polymer and a polylactide—(PLA) based polymer with a branched polyhydroxyalkanoate (PHA) homopolymer or a polylactide—(PLA) based polymer, the PHA homopolymer including (R)-3-hydroxybutyrate (3HB) units and (R)-3-hydroxyalkanoate (3HA) units having a structure of [(R)-3HB]ₓ—[(R)-3HA]₁₋ₓ, where x is the mol % of 3HB units in PHA homopolymer, the PHA of the copolymer including 3HB units and 3HA units having a structure of [(R)-3HB]_{xc}—[(R)-3HA]_{1-xc}, where x_{c} is the mol % of 3HB units in the PHA polymer of the copolymer, wherein the 3HA units of the PHA homopolymer and the 3HA units of the PHA polymer of the copolymer have side groups of at least three carbon atoms, and wherein the scaffold is radially expandable in a blood vessel of a patient.

The fourth set of embodiments may have one or more, or any combination of the following aspects (1) to (17): (1) wherein x is between 2% and 50%, (2) wherein x_{c} is between 2% and 50%, (3) wherein the number average molecular weight (Mn) of the copolymer is greater than 20 kDa, (4) wherein the Mn of the PLA-based polymer is greater than 50 kDa, (5) wherein the Mn of the PHA homopolymer is greater than 50 kDa, (6) wherein the 3HA units of the PHA homopolymer or the PHA of the copolymer are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd); (7) wherein the PLA-based polymer comprises 2% to 15% of D-lactide units, (8) wherein a mol % y_{c} of the PHA of the copolymer is 5 to 30 mol % of the copolymer, (9) wherein a mol % y_{c} of the PHA of the copolymer is 70 to 95 mol % of the copolymer, (10) wherein a wt % y of the PHA homopolymer is 5 to 30 wt % of the blend, (11) wherein a wt % of the PHA homopolymer is 70 to 95 wt % of the blend, (12) wherein a wt % z of the copolymer is 5 to 30 wt % of the blend, (13) wherein the wt % z of the copolymer is 70 to 95 wt % of the blend, and (14) wherein x is between 2% and 15%, (15) wherein the number of side groups is 3 to 7, (16) wherein a crystallinity of the blend is 20% to 50%, and (17) wherein a Young's modulus of the blend is greater than 500 MPa and a flexural modulus is 6 to 10 GPa.

### BRIEF DESCRIPTION OF THE DRAWINGS

FIG. 1 depicts a schematic view of a branched PHA system showing the backbone with medium chain length side groups.

FIG. 2 depicts a view of an exemplary scaffold.

### DETAILED DESCRIPTION OF THE INVENTION

In many treatment applications using stents, stents expand and hold open narrowed portions of blood vessels. As indicated, to achieve this the stent must possess a radial strength in an expanded state that is sufficiently high and sustainable to maintain the expanded vessel size for a period of weeks or months. This generally requires a high strength and rigid material. In the case of bioresorbable polymer stents or scaffolds, bioresorbable polymers that are stiff and rigid have been proposed and used in stents for coronary intervention. Such polymers are stiff or rigid under physiological conditions within a human body. These polymers tend to be semicrystalline polymers that have a glass transition temperature (Tg) in a dry state sufficiently above human body temperature (approximately 37° C.) that the polymer is stiff or rigid at these conditions.

Fabricating a vascular scaffold from such materials with sufficient fracture toughness or fracture resistance is challenging due to their brittle nature. Vascular scaffolds are subjected to deformation and stress during manufacture when crimped to a delivery diameter, when deployed from a delivery diameter to a deployment diameter, and during use after deployment. The vascular scaffolds are susceptible to fracture during manufacture, deployment, and use. In addition, stability of the properties of a scaffold made of such materials can limit the shelf life of scaffold based on such materials and is problem to be overcome. Specifically, properties such as strength and toughness tend to gradually change during storage at room temperature due to polymer chain rearrangement due to stress relaxation.

Embodiments of the invention relate to implantable medical devices such as stents including branched, in particular, sparse comb, polyhydroxyalkanoate (PHA) systems. The stent may include a stent body, scaffold, or substrate made partially or completely of polymer material including branched PHA. The stent body may also include a coating that includes a therapeutic agent.

Branched PHA systems provide increased ductility to a polymer without significant loss of strength. The incorporation of branched PHA systems in vascular scaffolds can reduce or eliminate problems associated with fracture toughness and stability during storage. Not to be limited by theory, it is believed that branched PHA achieves this through medium chain length (mcl) side groups that raise the thermodynamic barriers to amorphous phase chain rearrangement, thereby improving fracture toughness and expansion capability throughout shelf-life. Embodiments of branched PHA systems include scaffold including (1) branched PHA homopolymer; (2) homopolymer blend of branched PHA and polylactide (PLA)-based polymer; (3) copolymer of a branched PHA polymer and a PLA-based polymer; and (4) blend of a branched PHA homopolymer and a branched PHA/PLA copolymer. Unless otherwise specified, as used herein “PHA” refers to a branched PHA polymer.

The PHA systems of the scaffolds are based on a class of PHA copolymer which is a random copolymer having (R)-3-hydroxybutyrate (3HB) units and (R)-3-hydroxyalkanoate (3HA) units. The structure of the PHA copolymer is

[(R)-3HB]ₓ—[(R)-3HA]₁₋ₓ,

where x is the mol % of (R)-3HB units. The 3HA units have side groups including at least three carbon atoms, referred to as medium chain length (mcl) side groups. The value of x may be between 2 and 50%. The “3” in the “3HB” and “3HA” units reference to 3 carbon atoms, not including pendent methyl group or side chain carbons.

The molecular structure of the branched polyhydroxyalkanoate (PHA) copolymer is

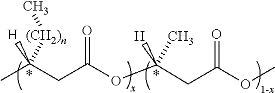

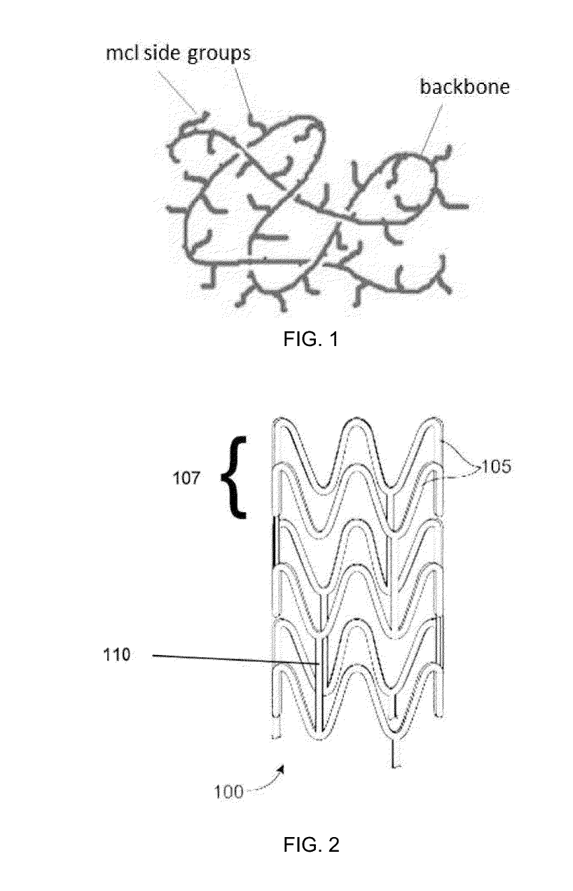

The number of carbon atoms (N) in the mcl side groups is n+1, where N is at least 3 so n is at least 2. Exemplary 3HA units with the (mcl) side groups include (R)-3-hydroxyhexanoate (3HHx) (n=2; N=3), (R)-3-hydroxyoctanoate (3HO) (n=4, N=5), (R)-3-hydroxydecanoate (3HD) (n=6; N=7), and (R)-3-hydroxyoctadecanoate (3HOd) (n=14; N=15). FIG. 1 depicts a schematic view of a branched PHA system showing the backbone with mcl side groups.

With the branching proportion, x, between 2 and 50%, as shown in FIG. 1, it is believed that the fracture toughness and ductility may be improved relative to a polymer such as poly(L-lactide) (PLLA), as the short branches on the PHA increase local molecular mobility in the amorphous phase through steric hindrance. Unless otherwise specified, a “PHA homopolymer” refers to a branched PHA copolymer with only HA units.

The fracture toughness and ductility are important in reducing material-level damage during crimping and in vitro/in vivo deployment of a bioresorbable scaffold. This translates into achieving a sufficiently high radial strength with a reduced strut cross-section, as described herein, and a reduction or prevention of fracture upon expansion of the scaffold from the crimped state.

Thermal, crystallinity, and mechanical properties of the PHA homopolymer depend upon the mole % of 3HA units and the number of carbon atoms in the mcl side groups. Thermal properties include the melting temperature (Tm) and glass transition temperature (Tg). Mechanical properties include the Young's modulus, flexural modulus, strength, and elongation at break. The mcl length and the mol % of the PHA system can be selected to provide desired thermal properties, crystallinity, and mechanical properties. The mcl length and the mol % of the PHA system can be selected to provide desired scaffold properties such as radial strength and radial stiffness.

Noda et al. (G.-Q. Chen (ed.), Plastics from Bacteria: Natural Functions and Applications, Microbiology Monographs, Vol. 14, DOI 10.1007/978-3-642-03287_{—}5_{—}10, p. 237-255) shows that the Tm of PHA copolymers decrease as the mol %, x, of the 3HA comonomer units increase, such as 3HHx, 3HO, and 3HD. Additionally, the degree of Tm lowering for a given mole percentage incorporation of comonomer is about the same for all mcl 3HA comonomers. The melt temperature varies between about 178° C. for x=0% to 100° C. for x of about 11 to 14 mol %. The mol % of 3HA units can be selected to obtain a Tm of the PHA homopolymer, for example, 100 to 110° C., 110 to 120° C., 100 to 120° C., 100 to 130° C., 130 to 140° C., 100 to 140° C., 100 to 150° C., 150 to 160° C., 160 to 170° C., or greater than 170° C.

Noda et al. shows that 3HA units with mcl side groups with at least 3 carbon atoms lower the crystallinity of PHA homopolymers. The crystallinity appears to be independent of side group size for mcl groups of at least three carbon atoms. For example, PHBHx and PHBO homopolymers, with propyl and pentyl side groups respectively, show a similar crystallinity-lowering trend. The crystallinity varies between about 55% for x=0% to about 20% for x of about 14 mol %.

Through the mol % of 3HA units, the crystallinity of the PHA homopolymer can be adjusted or selected to be 20 to 50%, 20 to 25%, 25 to 30%, 30 to 35%, 35 to 40%, 40 to 45%, and 45 to 50%.

Noda et al. further shows that the Tg varies with both mol % of 3HA groups and N. For x=8 mol %, the Tg varies from about 0° C. (N=3) to about −11° C. (N=7) and for x=12 mol %, the Tg varies from about −2° C. (N=3) to about −9° C. (N=5). Through either or both the mol % of 3HA and N, the Tg can be adjusted to be 2 to 0° C., 0 to −3° C., -3 to -6° C., −6 to −10° C., −10 to −15° C., −15 to −20° C., or less than −20° C.

The number average molecular weight (Mn) of PHA in a scaffold before or after sterilization may be 50 to 100 kDa, 50 to 60 kDa, 60 to 80 kDa, 80 to 100 kDa, greater than 50 kDa, or greater than 100 kDa.

Mechanical properties such as strength, stiffness, and ultimate elongation depend on both the mol % of 3HA units and N. Ultimate elongation increases with increasing N and mol % of 3HA. The strength and stiffness or modulus decreases as N increases and as mol % of 3HA increases. Through variation of the N, mol %, or both, the modulus of PHA can be adjusted or selected to be any of the values or ranges disclosed herein.

For each of the PHA system scaffold embodiments, the polymer of a scaffold may have a Young's modulus greater than 500 MPa, or more narrowly, 500 to 600 MPa, 600 to 700 MPa, or 700 to 1000 MPa. The polymer of a scaffold may have a flexular modulus of greater than 2.5 GPa, or more narrowly, 2.5 to 3 GPa, 3 to 5 GPa, 5 to 6 GPa, 6 to 10 GPa, 6 to 8 GPa, 8 to 10 GPa, or greater than 10 GPa. The properties of the PHA system of the scaffold can be adjusted with enhanced processing that are disclosed herein. The properties disclosed for PHA system scaffolds disclosed herein refer to the properties of the scaffold in a finished state, before or after sterilization.

Embodiments of the invention include a scaffold made substantially or completely of a PHA homopolymer. “Substantially” may correspondent to greater than 90 wt %, greater than 95 wt %, or greater than 99 wt %. The scaffold may have a PHA homopolymer composition of 90 to 95% or 95 to 99%. The scaffold may include other components that include, but are not limited to, fillers, plasticizers, visualization materials (e.g., radiopaque), or therapeutic agents.

The 3HA units have mcl side groups of at least 3 carbon atoms. The 3HA units may have mcl side groups having any number of carbon atoms between and including 3 and 18, or any number greater than 18. The mol % of 3HA units may be 2%≦x≦50% or in any of the ranges disclosed herein above for a PHA polymer.

In preferred embodiments, the mol % 3HA units, x, may be less than 15%. In this range, the PHA polymer of the scaffold may have sufficient stiffness to provide high radial stiffness while also having high ductility or fracture resistance. Exemplary ranges of x may include 2 to 20%, 2 to 15%, 2 to 15%, 2 to 13%, 2 to 4%, 4 to 6%, 6 to 8%, 8 to 10%, 10 to 15%, 5 to 15%, 7 to 15%, 5 to 13%, 7 to 13%, 8 to 12%, 9%, 11%, 15 to 20%, 18 to 20%, 20 to 40%, or 30 to 40%. In particular, the PHA polymer may be flexible/ductile between about 5% and 15% or from 7% and 13% and the PHA polymer may be stiff/brittle with x less than about 5% and soft/elastic with x greater than about 15%. Also, in preferred embodiments, N may be 3 to 7.

The PHA homopolymer may have any combination of the mol % of 3HA units and N disclosed herein above. The 3HA units may include 3HHx, 3HO, 3HD, or 3HOd.

Tm of the PHA homopolymer of the scaffold may be in any of the ranges disclosed herein above for a PHA polymer. A preferred Tm may be 120 to 160° C., 120 to 130° C., 130 to 140° C., 140 to 150° C., or 150 to 160° C. in order to allow for melt processing of the polymer.

The Tg of the PHA homopolymer of the scaffold may be 2 to 0° C., 0 to −3° C., −3 to −6° C., −6 to −10° C., −10 to −15° C., −15 to −20° C., or less than −20° C.

The crystallinity of the PHA homopolymer of the scaffold may be in any of the ranges disclosed herein above for a PHA copolymer. A preferred crystallinity may be greater than 20%, 20 to 50%, 20 to 30%, 30 to 40%, 30 to 55%, or 40 to 50%.

Further embodiments include a scaffold made from a combination of a branched PHA polymer and a PLA-based polymer. The combinations include a blend of PHA homopolymer and a PLA-based polymer, a copolymer of a branched PHA copolymer and a PLA-based polymer (PHA/PLA copolymer), and a blend of a PHA/PLA copolymer with a PHA homopolymer, a PLA-based polymer or both.

A PLA-based polymer includes poly(L-lactide), poly(D-lactide), poly(D,L-lactide), poly(D,L-lactide) having a constitutional unit weight-to-weight (wt/wt) ratio of about 96/4, poly(L-lactide-co-D,L-lactide), poly(L-lactide-co-glycolide), poly(D,L-lactide-co-glycolide), poly(L-lactide-co-caprolactone), poly(D,L-lactide-co-caprolactone), poly(D,L-lactide) made from meso-lactide, and poly(D,L-lactide) made from polymerization of a racemic mixture of L- and D-lactides. A PLA-based polymer can include a PLA with a D-lactide content greater than 0 mol % and less than 15 mol %, or more narrowly, 1 to 15 mol %, 1 to 5 mol %, 5 to 10%, or 10 to 15 mol %. The PLA-based polymers include poly(D,L-lactide) having a constitutional unit weight-to-weight (wt/wt) ratio of about 93/7, about 94/6, about 95/5, about 96/4, about 97/3, about 98/2, or about 99/1. The caprolactone copolymers may have 1 to 5 wt % caprolactone units. The term “unit” or “constitutional unit” refers to the composition of a monomer as it appears in a polymer.

Embodiments of the invention include a scaffold made substantially or completely of a blend of a PHA homopolymer and a PLA-based polymer. “Substantially” may correspondent to greater than 90 wt %, greater than 95 wt %, or greater than 99 wt %. The blend may be 90 to 95% or 95 to 99% of the scaffold. The scaffold may include other components that include, but are not limited to, fillers, plasticizers, visualization materials (e.g., radiopaque), or therapeutic agents.

The Mn of the PLA-based polymer or component of the scaffold may be greater than 50 kD. More narrowly, the Mn of the PLA-based polymer may be 50 to 150 kDa, 50 to 60 kDa, 60 to 70 kDa, 70 to 80 kDa, 80 to 90 kDa, 90 to 100 kDa, 100 to 120 kDa, or 120 to 150 kDa.

The Mn of the PHA homopolymer or component of the scaffold may be greater than 50 kD. More narrowly, the Mn of the PHA homopolymer may be 50 to 150 kDa, 50 to 60 kDa, 60 to 70 kDa, 70 to 80 kDa, 80 to 90 kDa, 90 to 100 kDa, 100 to 120 kDa, or 120 to 150 kDa.

The PHA homopolymer wt % proportion of the blend, y, may be between greater than 0 wt % and less than 100 wt %. More narrowly, the PHA homopolymer proportion may be 0.1 to 1 wt %, 0.1 to 2 wt %, 1 to 5 wt %, 5 to 10 wt %, 10 to 15 wt %, 15 to 25 wt %, 25 to 40 wt %, 40 to 50 wt %, 50 to 65 wt %, 65 to 75 wt %, 75 wt % to 85 wt %, 85 to 90 wt %, 90 to 95 wt %, 95 to 98 wt %, 95 to 99 wt %, or 95 to 99.9 wt %.

The PLA-based polymer wt % proportion of the blend, 1-y, may be between greater than 0 wt % and less than 100 wt %. More narrowly, the PLA-based polymer proportion may be 0.1 to 1 wt %, 0.1 to 2 wt %, 1 to 5 wt %, 5 to 10 wt %, 10 to 15 wt %, 15 to 25 wt %, 25 to 40 wt %, 40 to 50 wt %, 50 to 65 wt %, 65 to 75 wt %, 75 wt % to 85 wt %, 85 to 90 wt %, 90 to 95 wt %, 95 to 98 wt %, 95 to 99 wt %, or 95 to 99.9 wt %.

The 3HA units of the PHA homopolymer may have mcl units having an N that is any number between and including 3 and 18, or any number greater than 18. Preferably, N is 3 to 7.

The mol % of 3HA units relative to the PHA homopolymer may be 2%≦x≦50% and in any of the ranges disclosed herein above for a PHA polymer. In certain embodiments, for example, when the wt % y of PHA homopolymer in the blend is less than 50%, the mol % of 3HA units, x, may be less than 40%, 20 to 40%, 2 to 20%, 2 to 15%, 2 to 13%, 2 to 4%, 4 to 6%, 6 to 8%, 8 to 10%, 10 to 15%, 15 to 20%, or 18 to 20%.

The blend may have any combination of wt % of PHA homopolymer (y), the mol % of 3HA units (x), and N of 3HA units disclosed herein above. The 3HA units may include 3HHx, 3HO, 3HD, or 3HOd.

In some embodiments in which the blend has much higher PLA content, for example, in which 1-y is greater than 90 wt %, greater than 95 wt %, or greater than 98%, x may be 2 to 20%, N may be 3 to 7. In this case, the Mn of PLA (finished product) may be 55 to 150 kDa and Mn PHA (finished product) may be 30 to 70 kDa. The value of x of 2 to 20% may ensure or provide some compatibility between PLLA and PHA. The range of N of 3 to 7 may provide some ductility without significantly disrupting the ability of the materials to crystallize so the material can achieved preferred level of crystallinity. The molecular weight ranges may provide high potential for mixing and overall performance in the finished product with respect to material properties and degradation behavior.

In some embodiments in which the blend has a very low PLA content, for example, in which 1-y is less than 5 wt %, 1 wt %, or 0.5 wt %, x may be 2 to 10%, N may be 3 to 7. The intrinsic viscosity (IV) of the PLA resin used for processing may be greater than 5.0 dL/g. The Mn of PLA (finished product) may be greater than 150kDa. The IV of PHA (resin) used in processing may be 2.0 to 4.0 dL/g and the Mn of PHA (finished product) may be 55 to 155 kDa. A low percentage of higher molecular weight PLLA enhances nucleation of crystallites, crystallization, and melt stability. To limit disruption of crystallization, the mol % of 3HA units may be on the lower end, N of 3 to 7, which offers some ductility, but without too much disruption of crystallization.

In some embodiments in which the blend has an intermediate level of PLA, for example, in which 1-y is about 70% or 55 to 70 wt % or 65 to 75 wt %, x may be less than 2 to 10%, and N may be 3 to 7. The Mn PLA (finished product) may be 66 to 130 kDa and the Mn PHA (finished product) may be 66 to 130 kDa. The blend may have 2 to 10 wt % 3HA units, leading to modest disruption of crystallinity while imparting ductility due to an N of 3 to 7.

The thermal properties of the blend, mechanical properties of the blend, crystallinity of the blend, scaffold properties depend on parameters including the wt % of the PHA homopolymer (y), the mol % of the 3HA units (x), and the N of the PHA units. Any of the parameters or combination of parameters may be adjusted or selected to obtain any combination of blend or scaffold properties herein disclosed.

The Tm of blend may be 100 to 110° C., 110 to 120° C., 100 to 120° C., 100 to 130° C., 130 to 140° C., 100 to 140° C., 100 to 150° C., 150 to 160° C., 160 to 170° C., or greater than 170° C.

The Tg of the blend may be less than 10° C., 10 to 25° C., 25 to 37° C., 37 to 45° C., 40 to 45° C., 45 to 50° C., 50 to 55° C., or 60 to 65° C., or greater than 65° C.

The crystallinity of the blend or scaffold made of the blend may be 20 to 50%, 20 to 25%, 25 to 30%, 30 to 35%, 35 to 40%, 40 to 45%, and 45 to 50%.

In exemplary embodiments in which the blend is mostly PLA-based polymer, y is less than 50 wt %, 40 wt %, or 20 wt %; the N is greater 10; and x is 5 to 15 mol %.

In exemplary embodiments in which the blend is mostly PHA homopolymer, y is greater than 50 wt %, 60 wt %, or 80 wt %; the N is 3 to 8; and x is 3 to 12 mol %.

Embodiments of the invention include a scaffold made substantially or completely of a copolymer of a PHA polymer and a PLA-based polymer (PHA/PLA copolymer). “Substantially” may correspondent to greater than 90 wt %, greater than 95 wt %, or greater than 99 wt %. The copolymer may be 90 to 95% or 95 to 99% of the scaffold. The scaffold may include other components that include, but are not limited to, fillers, plasticizers, visualization materials (e.g., radiopaque), or therapeutic agents.

The PHA/PLA copolymer may include random or alternating lactide units, 3HA units and 3HB units. The PHA/PLA copolymer may also include glycolide units.

The Mn of the PHA/PLA copolymer may be greater than 50 kDa. More narrowly, the Mn of the PHA/PLA copolymer may be 50 to 150 kDa, 50 to 60 kDa, 60 to 70 kDa, 70 to 80 kDa, 80 to 90 kDa, 90 to 100 kDa, 100 to 120 kDa, or 120 to 150 kDa.

The PHA mol % (PHB and PHA units) of the PHA/PLA copolymer, y_{c}, may be between greater than 0 mol % and less than 100 mol %. More narrowly, the PHA copolymer proportion, y_{c}, may be 0.1 to 1 mol %, 0.1 to 2 mol %, 1 to 5 mol %, 5 to 10 mol %, 10 to 15 mol %, 15 to 25 mol %, 25 to 40 mol %, 40 to 50 mol %, 50 to 65 mol %, 65 to 75 mol %, 75 mol % to 85 mol %, 85 to 90 mol %, 90 to 95 mol %, 95 to 98 mol %, 95 to 99 mol %, or 95 to 99.9 mol %.

The 3HA units of the PHA/PLA copolymer may have mcl units having an Nc that is any number between and including 3 and 18, or any number greater than 18. The mol % of 3HA units relative to the 3HA and 3HB units may be 2%≦x_{c}≦50% and in any of the ranges disclosed herein above for a PHA polymer. In certain embodiments, for example, when the mol % y_{c} of PHA of the PHA/PLA copolymer is less than 50%, the mol % 3HA units relative to 3HA and 3HB units, x_{c}, may be 2 to 40%, 20 to 40%, 2 to 20%, less than 15%, 2 to 15%, 2 to 13%, 2 to 4%, 4 to 6%, 6 to 8%, 8 to 10%, 10 to 15%, or 15 to 20%, or 18 to 20%.

The PHA/PLA copolymer may have any combination of mol % of PHA polymer (y_{c}), the mol % of 3HA units (x_{c}), and Nc of 3HA units disclosed herein above. The 3HA units may include 3HHx, 3HO, 3HD, or 3HOd.

In some embodiments in which the PHA content of the copolymer is high, for example, in which yc is greater than 90, 95%, or 98%, the xc is less than 10% and Nc of 3 to 7. The PHA/PLA copolymer Mn (finished product) maybe 66-130 kDa. The low content PLA may provide some disruption of PHA crystallization, similar to the disruption provide by 3HA units.

In some embodiments in which the PLA content of the copolymer is high, for example, in which yc is less than 10%, 5%, or 2%, the xc is 2 to 40%, 10 to 20%, or 20 to 40% and Nc of 3 to 7. The PHA/PLA copolymer Mn (finished product) maybe 66 to 130 kDa. In these embodiments, the 3HA units are more sparsely distributed throughout the chain to add ductility to a largely PLLA matrix that should still readily crystallize for provide strength.

The thermal properties of the PHA/PLA copolymer, mechanical properties of the PHA/PLA copolymer, crystallinity of the PHA/PLA copolymer, scaffold properties depend on parameters including the mol % of the PHA (y_{c}), the mol % of the 3HA units (x_{c}), and the Nc of the PHA units. Any of the parameters or combination of parameters may be adjusted or selected to obtain any combination of PHA/PLA copolymer or scaffold properties herein disclosed.

The Tm of PHA/PLA copolymer may be 100 to 110° C., 110 to 120° C., 100 to 120° C., 100 to 130° C., 130 to 140° C., 100 to 140° C., 100 to 150° C., 150 to 160° C., 160 to 170° C., or greater than 170° C.

The Tg of the PHA/PLA copolymer may be less than 10° C., 10 to 25° C., 25 to 37° C., 37 to 45° C., 40 to 45° C., 45 to 50° C., 50 to 55° C., or 60 to 65° C., or greater than 65° C.

The crystallinity of the PHA/PLA copolymer or scaffold made of the PHA/PLA copolymer may be 20 to 50%, 20 to 25%, 25 to 30%, 30 to 35%, 35 to 40%, 40 to 45%, and 45 to 50%.

In exemplary embodiments in which the PHA/PLA copolymer is mostly PLA-based polymer, y_{c} is less than 50 mol %, 40 mol %, or 20 mol %; the Nc is greater 10; and x_{c} is 5 to 15 mol %.

In exemplary embodiments in which the PHA/PLA copolymer is mostly PHA copolymer, y_{c} is greater than 50 mol %, 60 mol %, or 80 mol %; the Nc is 3 to 8; and x_{c} is 3 to 12 mol %.

Embodiments of the invention include a scaffold made substantially or completely of a blend of a PHA/PLA copolymer with a PHA homopolymer or a PLA-based polymer. The PHA/PLA copolymer is a copolymer of a PHA polymer and a PLA-based polymer. “Substantially” may correspondent to greater than 90 wt %, greater than 95 wt %, or greater than 99 wt %. The blend may be 90 to 95% or 95 to 99% of the scaffold. The scaffold may include other components that include, but are not limited to, fillers, plasticizers, visualization materials (e.g., radiopaque), or therapeutic agents.

For a PHA homopolymer and PHA/PLA copolymer blend, the PHA homopolymer wt % proportion of the blend, y, may be between greater than 0 wt % and less than 100 wt %. More narrowly, the PHA homopolymer proportion, y, may be 0.1 to 1 wt %, 0.1 to 2 wt %, 1 to 5 wt %, 5 to 10 wt %, 10 to 15 wt %, 15 to 25 wt %, 25 to 40 wt %, 40 to 50 wt %, 50 to 65 wt %, 65 to 75 wt %, 75 wt % to 85 wt %, 85 to 90 wt %, 90 to 95 wt %, 95 to 98 wt %, 95 to 99 wt %, or 95 to 99.9 wt %.

For a PLA-based polymer and PHA/PLA copolymer blend, the PLA-based polymer wt % proportion of the blend, w, may be between greater than 0 wt % and less than 100 wt %. More narrowly, the PLA-based polymer proportion, y, may be 0.1 to 1 wt %, 0.1 to 2 wt %, 1 to 5 wt %, 5 to 10 wt %, 10 to 15 wt %, 15 to 25 wt %, 25 to 40 wt %, 40 to 50 wt %, 50 to 65 wt %, 65 to 75 wt %, 75 wt % to 85 wt %, 85 to 90 wt %, 90 to 95 wt %, 95 to 98 wt %, 95 to 99 wt %, or 95 to 99.9 wt %.

For either blend, the PHA/PLA copolymer wt % proportion of the blend, z, may be between greater than 0 wt % and less than 100 wt %.

The PHA/PLA copolymer wt % proportion of the blend, z, may be between greater than 0 wt % and less than 100 wt %. More narrowly, the PHA/PLA copolymer proportion, z, may be 0.1 to 1 wt %, 0.1 to 2 wt %, 1 to 5 wt %, 5 to 10 wt %, 10 to 15 wt %, 15 to 25 wt %, 25 to 40 wt %, 40 to 50 wt %, 50 to 65 wt %, 65 to 75 wt %, 75 wt % to 85 wt %, 85 to 90 wt %, 90 to 95 wt %, 95 to 98 wt %, 95 to 99 wt %, or 95 to 99.9 wt %.

Exemplary embodiments of a PHA homopolymer and PHA/PLA copolymer blend may have a relatively high PHA homopolymer proportion, e.g., y greater than 90 wt %, greater than 95 wt %, or greater than 98 wt %. The xc may 2 to 10% and the N and the Nc may be 3 to 7. The PHA/PLA copolymer Mn (finished product) may be 66 to 130 kDa. The IV of PHA (resin) used in processing may be 2.0 to 4.0 dL/g and the Mn of PHA homopolymer (finished product) may be 55 to 155 kDa.

Exemplary embodiments of a PLA-based polymer and PHA/PLA copolymer blend may have a relatively high PLA-based polymer proportion, e.g., w greater than 90 wt %, greater than 95 wt %, or greater than 98 wt %. The x_{c} may be 2 to 40%, 10 to 20%, or 20 to 40% and Nc may be 3 to 7. The PHA/PLA copolymer Mn (finished product) maybe 66 to 130 kDa. The Mn of PLA (finished product) may be 55 to 150 kDa.

The scaffold may include any combination of blend component compositions disclosed herein.

The PHA/PLA copolymer may include random or alternating lactide units, 3HA units, and 3HB units. The PHA/PLA copolymer may also include glycolide units.

The Mn of the PHA/PLA copolymer may be greater than 20 kDa. More narrowly, the Mn of the PHA/PLA copolymer may be 20 to 120 kDa, 20 to 30 kDa, 30 to 40 kDa, 40 to 50 kDa, 50 to 60 kDa, 60 to 70 kDa, 70 to 90 kDa, or 90 to 120 kDa.

The Mn of the PLA-based polymer or component may be greater than 50 kD. More narrowly, the Mn of the PLA-based polymer may be 50 to 150 kDa, 50 to 60 kDa, 60 to 70 kDa, 70 to 80 kDa, 80 to 90 kDa, 90 to 100 kDa, 100 to 120 kDa, or 120 to 150 kDa.

The Mn of the PHA homopolymer or component of the scaffold may be greater than 50 kD. More narrowly, the Mn of the PHA homopolymer may be 50 to 150 kDa, 50 to 60 kDa, 60 to 70 kDa, 70 to 80 kDa, 80 to 90 kDa, 90 to 100 kDa, 100 to 120 kDa, or 120 to 150 kDa.

The 3HA units of the PHA homopolymer may have mcl units having an N that is any number between and including 3 and 18, or any number greater than 18. The mol % of 3HA units relative to the PHA homopolymer may be 2%≦x≦50% and in any of the ranges disclosed herein above for a PHA homopolymer. In certain embodiments, for example, when the wt % y of PHA homopolymer in the blend is less than 50%, the mol % 3HA units, x, may be 2 to 40%, 20 to 40%, 2 to 20%, 2 to 15%, 2 to 13%, 2 to 4%, 4 to 6%, 6 to 8%, 8 to 10%, 10 to 15%, or 15 to 20%, or 18 to 20%.

The PHA copolymer mol % (3HB and 3HA units) of the PHA/PLA copolymer, y_{c}, (relative to the PHA/PLA copolymer only) may be between greater than 0 mol % and less than 100 mol %. More narrowly, the PHA copolymer proportion, y_{c}, may be 0.1 to 1 mol %, 0.1 to 2 mol %, 1 to 5 mol %, 5 to 10 mol %, 10 to 15 mol %, 15 to 25 mol %, 25 to 40 mol %, 40 to 50 mol %, 50 to 65 mol %, 65 to 75 mol %, 75 mol % to 85 mol %, 85 to 90 mol %, 90 to 95 mol %, 95 to 98 mol %, 95 to 99 mol %, or 95 to 99.9 mol %.

The 3HA units of the PHA/PLA copolymer may have mcl units having an Nc that is any number between and including 3 and 18, or any number greater than 18. The mol % of 3HA units relative to the 3HA and 3HB units may be 2%≦x_{c}≦50% and in any of the ranges disclosed herein above for a PHA homopolymer. In certain embodiments, for example, when the mol % y_{c} of PHA copolymer in the PHA/PLA copolymer is less than 50%, the mol % 3HA units relative to 3HA and 3HB units, x_{c}, may be 2 to 40%, 20 to 40%, 2 to 20%, 2 to 15%, 2 to 13%, 2 to 4%, 4 to 6%, 6 to 8%, 8 to 10%, 10 to 15%, 15 to 20%, or 18 to 20%.

The blend of the PHA homopolymer, and PHA/PLA copolymer of the blend may have any combination of wt % of PHA homopolymer (y), the mol % of 3HA units (x) in the PHA homopolymer, N of 3HA units in the PHA homopolymer, mol % of PHA/PLA copolymer (z), mol % of PHA in the PHA/PLA copolymer (y_{c}), the mol % of 3HA units (x_{c}), and Nc of 3HA units disclosed herein above. The 3HA units of the PHA homopolymer and PHA/PLA copolymer may include 3HHx, 3HO, 3HD, or 3HOd.\

The blend of the PLA-based polymer and PHA/PLA copolymer of the blend may have any combination of wt % of PLA-based polymer (w), mol % of PHA/PLA copolymer (z), mol % of PHA in the PHA/PLA copolymer (y_{c}), the mol % of 3HA units (x_{c}), and Nc of 3HA units disclosed herein above.

The thermal properties of the blend, mechanical properties of the blend, crystallinity of the blend, scaffold properties depend on parameters including the wt % of PHA homopolymer (y), the mol % of 3HA units (x) in the PHA homopolymer, wt % of PLA-based polymer (w), N of 3HA units in the PHA homopolymer, mol % of PHA/PLA copolymer (z), mol % of PHA in the PHA/PLA copolymer (y_{c}), the mol % of 3HA units (x_{c}), and Nc of 3HA units. Any of the parameters or combination of parameters may be adjusted or selected to obtain any combination of blend or scaffold properties herein disclosed.

The Tm of the blend may be 100 to 110° C., 110 to 120° C., 100 to 120° C., 100 to 130° C., 130 to 140° C., 100 to 140° C., 100 to 150° C., 150 to 160° C., 160 to 170° C., or greater than 170° C.

The Tg of the blend may be less than 10° C., 10 to 25° C., 25 to 37° C., 37 to 45° C., 40 to 45° C., 45 to 50° C., 50 to 55° C., or 60 to 65° C., or greater than 65° C.

The crystallinity of the blend or scaffold made of the blend may be 20 to 50%, 20 to 25%, 25 to 30%, 30 to 35%, 35 to 40%, 40 to 45%, and 45 to 50%.

The various embodiments of the device may be configured to eventually completely absorb from an implant site. The device may provide drug delivery once implanted, provide mechanical support to the vessel, and then gradually completely absorb away. The device may also be configured to provide no mechanical support to a vessel and serve primarily as a drug delivery vehicle. The device may be configured to completely erode away within 6 months, 6 to 12 months, 12 to 18 months, 18 months to 2 years, or greater than 2 years.

A completely bioresorbable device may still include some nonbiodegradable elements such as radiopaque markers or particulate additives. The polymers of the device can be biostable, bioresorbable, bioabsorbable, biodegradable, or bioerodable. Biostable refers to polymers that are not biodegradable. The terms biodegradable, bioresorbable, bioabsorbable, and bioerodable are used interchangeably and refer to polymers that are capable of being completely degraded and/or eroded into different degrees of molecular levels when exposed to bodily fluids such as blood and can be gradually resorbed, absorbed, and/or eliminated by the body. The processes of breaking down and absorption of the polymer can be caused by, for example, by hydrolysis and metabolic processes.

A scaffold may have a tendency to decrease in diameter or recoil (e.g., 2 to 10%) right after implantation (i.e., less than about 30 minutes post-implantation) as well as over a period of days, weeks, or months. Once implanted, the device may not have radial strength sufficient to reduce or prevent the immediate or long-term recoil.

In some embodiments, the radial strength can be high enough to provide mechanical support to a vessel after expanding the vessel to an increase diameter or prevent or reduce a decrease in the diameter of the vessel. In such embodiments, the radial strength can be greater than 200 mm Hg, 200 to 250 mm Hg, 200 to 300 mm Hg, or higher than 300 mm Hg.

The mechanical properties of the scaffold material including PHA homopolymer; PHA/PLA-based polymer blend; PHA/PLA copolymer; PHA/PLA copolymer blend with PHA homopolymer, PLA-based polymer, or both may include elongation at break (ultimate elongation), tensile modulus, and strength. The scaffold polymer or material may have an elongation at break less than 3%, 3% to 15%, 3 to 5%, 3 to 6%, 6 to 10%, 10 to 12%, 12 to 15%, 15 to 20%, or greater than 20% at 25 deg C., 37° C., or in a range of 25 to 37° C. in a dry state or in a wet state. The scaffold polymer or material may have a tensile modulus less than 100 MPa, 100 to 2600 Mpa, 100 to 200 MPa, 200 to 400 MPa, 400 to 600 MPa, 600 to 800 MPa, 800 to 1000 MPa, 1000 to 1200 MPa, 1200 to 1400 MPa, 1400 to 1600 MPa, 1600 to 1800 MPa, 1800 to 2000 MPa, 2000 to 2200 MPa, 2200 to 2400 MPa, 2400 to 2600 MPa, or greater than 2600 MPa at 25 deg C., 37° C., or in a range of 25 to 37° C. in a dry state or in a wet state. The wet state may correspond to soaking the material for at least 2 minutes in a simulated body fluid such as a phosphate buffered saline solution.

Drug delivery from the device can be provided from a coating on a surface of the stent body of the device. The coating may be in the form a neat drug. Alternatively, the coating may include a polymer matrix with the drug mixed or dissolved in the polymer. The polymer matrix can be bioresorbable. Suitable polymers for the drug delivery polymer can include any PLA-based polymer disclosed herein, any other polymers disclosed herein, and copolymers and blends thereof in any combination.

The coating can be formed by mixing the polymer and the drug in a solvent and applying the solution to the surface of the device. The drug release rate may be controlled by adjusting the ratio of drug and polymeric coating material. The drug may be released from the coating over a period of one to two weeks, up to one month, one to three months, one to four months, up to three months, or up to four months after implantation. Thickness of the coating on the device body may 1 to 20 microns, 1 to 2 microns, 1 to 5 microns, 2 to 5 microns, 3 to 5 microns, 5 to 10 microns, or 10 to 20 microns. In some embodiments, the stent body of the device includes a drug release coating and the body is free of drug, aside from any incidental migration of drug into the body from the coating. The Mn of the coating polymer may be less than 40 kDa, 40 to 60 kDa, 60 to 80 kDa, 80 to 100 kDa.

Alternatively or additionally, the drug can also be embedded or dispersed into the body of device, and be slowly released up to months (e. g., one to three months or three to six months after implantation) and while the device is degrading. In this case, the drug can be included with the polymer when the tube is formed that is used to form the device. For example, the drug can be included in the polymer melt during extrusion or injection molding or in a solution when the tube is formed from dipping or spraying or casting.

The final device can be balloon expandable or self expandable. In the case of a balloon expandable device, the geometry of the device can be an open-cell structure similar to the stent patterns disclosed herein or closed cell structure, each formed through laser cutting a hollow thin-walled tube.

In a balloon expandable device, when the device is crimped from a fabricated diameter to a crimped or delivery diameter onto a balloon, structural elements plastically deform. The device may have minimal recoil outward so the delivery diameter may different slightly from the crimped diameter. Aside from this minimal recoil, the device retains a crimped or delivery diameter without an inward force on the balloon due to the plastically deformed structural elements.

The device is radially expandable at, for example, 37° C. in body fluid or simulated body fluid. When the device is expanded by a balloon, the structural elements plastically deform. The device is expanded to an intended expansion or deployment diameter and retains the intended expansion diameter or a diameter slightly less due to acute recoil inward due to inward pressure from the vessel during the about the first 30 minutes. The diameter may vary slightly after the acute period due to biological interactions with the vessel, stress relaxation, or both. At the final expanded diameter, the device does not exert any chronic outward force, which is a radial outward force exerted by the device in excess of the radial inward force exerted by the vessel on device.

In the case of a self expandable device, when the device is compressed from a fabricated diameter to a delivery diameter on a balloon, the structural elements deform elastically. Therefore, to retain the device at the delivery diameter, the device is restrained in some manner with an inward force, for example with a sheath or a band. The compressed device is expanded to an intended expansion or deployment diameter by removing the inward restraining force which allows the device to self-expand to the intended deployment diameter. The structural elements deform elastically as the device self-expands. If the final expansion diameter is the same as the fabricated diameter, the device does not exert any chronic outward force. If the final expansion diameter is less than the fabricated diameter, the device does exert a chronic outward force.

The geometric structure of the device is not limited to any particular stent pattern or geometry. The device can have the form of a tubular scaffold structure that is composed of a plurality of ring struts and link struts. The ring struts form a plurality of cylindrical rings arranged about the cylindrical axis. The rings are connected by the link struts. The scaffold comprises an open framework of struts and links that define a generally tubular body with gaps in the body defined by the rings and struts.

This open framework of struts and links may be formed from a thin-walled cylindrical tube by a laser cutting device that cuts such a pattern into the thin-walled tube that may initially have no gaps in the tube wall. The scaffold may also be fabricated from a sheet by rolling and bonding the sheet to form the tube.

FIG. 2 depicts a view of an exemplary scaffold 100 which includes a pattern or network of interconnecting structural elements 105. FIG. 2 illustrates features that are typical to many stent patterns including cylindrical rings 107 connected by linking elements 110. The cylindrical rings are load bearing in that they provide radially directed force in response to an inward force on the scaffold. The linking elements generally function to hold the cylindrical rings together. Exemplary scaffolds are disclosed in US 2008/0275537, US 2011/0190872, and US 2011/0190871.

A stent or scaffold may have lengths of between 12 and 18 mm, 18 and 36 mm, 36 and 40 mm or even between 40 and 200 mm as fabricated or when implanted in an artery. Exemplary lengths include 12 mm, 14 mm, 18 mm, 24 mm, or 48 mm. The scaffold may have a pre-crimping or as-fabricated diameter of 2 to 3 mm, 2.5 to 3.5 mm, 3 to 4 mm, 3 to 5 mm, 5 to 10 mm, 6 to 8 mm, or any value between and including these endpoints. Diameter may refer to the inner diameter or outer diameter of the scaffold. Exemplary diameters include 2.5 mm, 3.0 mm, 3.25 mm, 3.5 mm, 4 mm, 5 mm, or 6 mm. The struts of the scaffold may have a radial wall thickness or width of 150 microns, 80 to 100 microns, 100 to 150 microns, 150 to 200 microns, 200 to 250 microns, 250 to 300 microns, 300 to 350 microns, 350 to 400 microns, or greater than 400 microns. Any combination of these ranges for radial wall thickness and width may be used.

The scaffold may be configured for being deployed by a non-compliant or semi-compliant balloon from a delivery diameter of 0.8 to 1 mm, 1 to 1.2 mm, 1.2 to 1.4 mm, 1.4 to 1.6 mm, 1.6 to 1.8 mm, and 1.8 to 2.2 mm, 1 mm, 1.2 mm, 1.3 mm, 1.4, mm, 1.6 mm, 1.8 mm, or 2 mm. Exemplary balloon sizes include 2.5 mm, 3 mm, 3.5 mm, 4 mm, 5.5 mm, 5 mm, 5.5 mm, 6 mm, 6.5 mm, 7 mm, or 8 mm, where the balloon size refers to a nominal inflated or deployment diameter of the balloon. The scaffold may be deployed to a diameter of between 2.5 mm and 3 mm, 3 mm and 3.5 mm, 3.5 mm and 4 mm, 4 mm and 10 mm, 7 and 9 mm, or any value between and including the endpoints. Embodiments of the invention include the scaffold in a crimped or delivery diameter over and in contact with a deflated catheter balloon.

The intended deployment diameter may correspond to, but is not limited to, the nominal deployment diameter of a catheter balloon which is configured to expand the scaffold. A device scaffold may be laser cut from a tube (i.e., a pre-cut tube) that is less than an intended deployment diameter. In this case, the pre-cut tube diameter may be 0.7 to 1 times the intended deployment diameter or any value in between and including the endpoints.

A device scaffold may be laser cut from a tube (i.e., a pre-cut tube) that is greater than an intended deployment diameter. In this case, the pre-cut tube diameter may be 1 to 1.5 times the intended deployment diameter, or any value in between and including the endpoints.

The device of the present invention may have a selected high crush recovery and crush resistance. Crush recovery describes the recovery of a tubular device subjected to a pinch or crush load. Scaffolds having a high crush recovery are particularly useful for treatment of the superficial femoral artery since upon implantation a scaffold is subjected to high crushing forces. The crush recovery can be described as the percent recovery to the device pre-crush shape or diameter from a certain percent crushed shape or diameter. Crush resistance is the minimum force required to cause a permanent deformation of a scaffold. The crush recovery and crush resistance can be based on a pre-crush shape or diameter of an as-fabricated device prior to crimping and expansion or a device after it has been crimped and expanded to an intended deployment diameter. The crush recovery of the device can be such that the device attains greater than about 70%, 80% or 90% of its diameter after being crushed to at least 50% of its pre-crush diameter.

The crush recovery and crush resistance of a balloon expandable scaffold that undergoes plastic deformation when crimped and deployed depend both on the scaffold material and scaffold pattern. Exemplary crush recoverable balloon expandable scaffold patterns can be found in US 2011/0190872 and US 2014/0067044.

The fabrication of a stent of the present invention can include: forming a hollow, thin-walled polymeric tube (i.e., pre-cut tube), preferably with no holes in the walls; processing that increases the strength of the scaffold body and also the radial strength of the scaffold, forming a stent scaffolding from the tube by laser machining a stent pattern in the tube; optionally forming a therapeutic coating over the scaffolding; crimping the stent over a delivery balloon, and sterilization the scaffold using radiation or an ethylene oxide process. Detailed discussion of the manufacturing processes of a bioabsorbable stent can be found elsewhere, e.g., U.S. Patent Publication Nos. 2007/0283552 and 2012/0073733.

A pre-cut tube can be formed by a melt processing method, a solution processing method, or a combination of both. Melt processing methods include extrusion and injection molding. In extrusion, for example, a polymer is processed in an extruder above the melting temperature of the polymer and forced through a die to form a tube. Solution processing methods include dipping (see e.g., US 2009/0319028) or spraying. A tube can also be formed by gel extrusion which includes extrusion of a polymer dissolved in a solvent.

The fabrication of the scaffold can include processing that increases the strength of the scaffold material and also the radial strength of the scaffold. The processing may increase the crystallinity of the scaffold polymer which increases the strength and stiffness of the scaffold material as the radial strength and radial stiffness of the scaffold. In another embodiment, the processing may increase the alignment of the scaffold polymer chains in the circumferential direction, axial direction, or both which increases the strength and radial strength of the scaffold. The processing can be performed prior to laser cutting, after laser cutting, or both.

The processing can include annealing the pre-cut tube and/or the scaffold at a temperature and for a time sufficient to increase the crystallinity to a desired level. The temperature may be between the glass transition temperature (Tg) of the scaffold polymer and the melting temperature (Tm) of the scaffold polymer.

Additionally or alternatively, the processing can include radially deforming the pre-cut tube to increase the radial strength of the tube (see e.g., US 2011/0066222). The radially expanded tube may then be laser cut to form a scaffold. The radial expansion increases the radial strength both through an increase in crystallinity and induced polymer chain alignment in the circumferential direction. The radial expansion process may be performed by a process such as blow molding. In blow molding, the pre-cut tube may be disposed within a mold and heated to a temperature between Tg and Tm and expanded by increasing a pressure inside of the tube.

The crystallinity of the pre-cut tube or scaffold prior to the processing may be less than 5%, 1 to 5%, 5 to 10%, less than 10%, 10 to 15%, less than 30%, or 15 to 30%. In an embodiment, the crystallinity prior to processing can be between 10-25%. The crystallinity of the processed tube, cut scaffold, crimped scaffold, sterilized scaffold, may be 20 to 30%, 20 to 25%, 30 to 40%, 40 to 45%, 45 to 50%, and greater than 50%.

A coating may be formed over the scaffold by mixing a coating polymer (e.g., a PLA-based polymer) and a drug (e.g., a macrocyclic drug) in a solvent and applying the solution to the surface of the scaffold. The application may be performed by spraying, dipping, ink jet printing, or rolling the scaffold in the solution. The coating may be formed as a series of layers by spraying or dipping followed by a step to remove all or most of residual solvent via, for example, evaporation by heating. The steps may then be repeated until a desired coating thickness is achieved.

The drug release rate may be controlled by adjusting the ratio of drug and polymeric coating material. The drug to polymer ration may be between 5:1 to 1:5. The drug may be released from the coating over a period of one to two weeks, up to one month, or up to three months after implantation. Thickness or average thickness of the coating on the device body may be less than 4 microns, 3 microns, 2.5 microns, 1 to 20 microns, 1 to 2 microns, 2 to 3 microns, 2 to 2.9 microns, 2 to 2.5 microns,1 to 5 microns, 2 to 5 microns, 3 to 5 microns, 5 to 10 microns, or 10 to 20 microns. The coating may be over part of the surface or the entire surface of a scaffold substrate. In some embodiments, the body of the device includes a drug release coating and the body is free of drug, aside from any incidental migration of drug into the body from the coating.

In some embodiments, the coating may include a primer layer between the scaffold body or structure and a drug delivery coating layer to enhance the adhesion of the drug coating to the scaffold. Alternatively, the coating may have no primer layer and only a drug delivery coating layer.

The coated scaffold may then be crimping over a delivery balloon. The crimped scaffold may then be packaged and then sterilized with radiation such as electron-beam (E-Beam) radiation or a low temperature ethylene oxide process (see e.g., US 2013/0032967). The range of E-beam exposure may be between 20 and 30 kGy, 25 to 35 kGy, or 25 to 30 kGy.

The device body may include or may be coated with one or more therapeutic agents, including an antiproliferative, anti-inflammatory or immune modulating, anti-migratory, anti-thrombotic or other pro-healing agent or a combination thereof. The anti-proliferative agent can be a natural proteineous agent such as a cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I1, actinomycin X1, and actinomycin C1), all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof.

The anti-inflammatory agent can be a steroidal anti-inflammatory agent, a nonsteroidal anti-inflammatory agent, or a combination thereof. In some embodiments, anti-inflammatory drugs include, but are not limited to, novolimus, myolimus, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, and genetically engineered epithelial cells. The foregoing substances are listed by way of example and are not meant to be limiting. Other active agents which are currently available or that may be developed in the future are equally applicable.

“Molecular weight” refers to either number average molecular weight (Mn) or weight average molecular weight (Mw). References to molecular weight (MW) herein refer to either Mn or Mw, unless otherwise specified. The Mn may be as measured by GPC-RI relative to a polystyrene standard.

“Semi-crystalline polymer” refers to a polymer that has or can have regions of crystalline molecular structure and amorphous regions. The crystalline regions may be referred to as crystallites or spherulites which can be dispersed or embedded within amorphous regions.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through movement. Tg of a given polymer can be dependent on the heating rate and can be influenced by the thermal history of the polymer as well as its degree of crystallinity. Furthermore, the chemical structure of the polymer heavily influences the glass transition by affecting mobility.

The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20° C./min heating rate.

The “melting temperature” (Tm) is the temperature at which a material changes from solid to liquid state. In polymers, Tm is the peak temperature at which a semicrystalline phase melts into an amorphous state. Such a melting process usually takes place within a relative narrow range (<20° C.), thus it is acceptable to report Tm as a single value.

“Elastic deformation” refers to deformation of a body in which the applied stress is small enough so that the object retains, substantially retains, or moves towards its original dimensions once the stress is released.

The term “plastic deformation” refers to permanent deformation that occurs in a material under stress after elastic limits have been exceeded.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to a change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Strength” refers to the maximum stress along an axis which a material will withstand prior to fracture. The ultimate strength is calculated from the maximum load applied during the test divided by the original cross-sectional area.

“Modulus” and “stiffness” may be defined as the ratio of a component of stress or force per unit area applied to a material divided by the strain along an axis of applied force that results from the applied force. The modulus or the stiffness typically is the initial slope of a stress-strain curve at low strain in the linear region. For example, a material has both a tensile and a compressive modulus.

The tensile stress on a material may be increased until it reaches a “tensile strength” which refers to the maximum tensile stress which a material will withstand prior to fracture. The ultimate tensile strength is calculated from the maximum load applied during a test divided by the original cross-sectional area. Similarly, “compressive strength” is the capacity of a material to withstand axially directed pushing forces. When the limit of compressive strength is reached, a material is crushed.

“Elongation at break” or “ultimate elongation” is the elongation recorded at the moment of rupture of a specimen in a tensile elongation test, expressed as a percentage of the original length or the strain.

“Toughness” is the amount of energy absorbed prior to fracture, or equivalently, the amount of work required to fracture a material. One measure of toughness is the area under a stress-strain curve from zero strain to the strain at fracture. The units of toughness in this case are in energy per unit volume of material. See, e.g., L. H. Van Vlack, “Elements of Materials Science and Engineering,” pp. 270-271, Addison-Wesley (Reading, Pa., 1989).

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A stent comprising:
a scaffold comprising a branched polyhydroxyalkanoate (PHA) polymer that is a random copolymer having (R)-3-hydroxybutyrate (3HB) units and (R)-3-hydroxyalkanoate (3HA) units with a structure of [(R)-3HB]ₓ—[(R)-3HA]₁₋ₓ, where x is the mol % of (R)-3HB units,
wherein the 3HA units have side groups of at least three carbon atoms,
wherein the scaffold is radially expandable in a blood vessel of a patient.

2. The stent of claim 1, wherein x is between 2% and 50%.

3. The stent of claim 1, wherein the number average molecular weight (Mn) of the PHA polymer is greater than 50 kDa.

4. The stent of claim 1, wherein the 3HA units are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd).

5. The stent of claim 1, wherein x is between 2% and 15% and the number of side groups to 3 to 7.

6. A stent comprising:
a scaffold comprising a blend of a polylactide—(PLA) based polymer and a branched polyhydroxyalkanoate (PHA) homopolymer,
the PHA homopolymer including (R)-3-hydroxybutyrate (3HB) units and (R)-3-hydroxyalkanoate (3HA) units having a structure of [(R)-3HB]ₓ—[(R)-3HA]₁₋ₓ, where x is the mol % of (R)-3HB units,
wherein the 3HA units have side groups of at least three carbon atoms,
wherein the scaffold is radially expandable in a blood vessel of a patient.

7. The stent of claim 6, wherein x is between 2% and 50%.

8. The stent of claim 6, wherein the number average molecular weight (Mn) of the PHA polymer is greater than 50 kDa.

9. The stent of claim 6, wherein the number average molecular weight (Mn) of the PLA-based polymer is greater than 50 kDa.

10. The stent of claim 6, wherein the 3HA units are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd).

11. The stent of claim 6, wherein the PLA-based polymer comprises to 15mol % of D-lactide units.

12. The stent of claim 6, wherein a wt % y of the PHA polymer is 5 to 30 wt % of the blend.

13. The stent of claim 6, wherein a wt % y of the PHA polymer is 70 to 95 wt % of the blend.

14. A stent comprising:
a scaffold comprising a copolymer of a polylactide—(PLA) based polymer and a branched polyhydroxyalkanoate (PHA) polymer,
the PHA polymer including (R)-3-hydroxybutyrate (3HB) units and (R)-3-hydroxyalkanoate (3HA) units having a structure of [(R)-3HB]_{xc}—[(R)-3HA]_{1-xc}, where x_{c} is the mol % of (R)-3HB units,
wherein the 3HA units have side groups including at least three carbon atoms,
wherein the scaffold is radially expandable in a blood vessel of a patient.

15. The stent of claim 14, wherein x_{c} is between 2% and 50%.

16. The stent of claim 14, wherein the number average molecular weight (Mn) of the PHA/PLA copolymer is greater than 50 kDa.

17. The stent of claim 14, wherein the 3HA units are selected from the group consisting of (R)-3-hydroxyhexanoate (3HHx), (R)-3-hydroxyoctanoate (3HO), (R)-3-hydroxydecanoate (3HD), and (R)-3-hydroxyoctadecanoate (3HOd).

18. The stent of claim 14, wherein the PLA-based polymer comprises 1 to 15mol % of D-lactide units.

19. The stent of claim 14, wherein a mol % y_{c} of the PHA is 5 to 30 mol % of the PHA/PLA copolymer.

20. The stent of claim 14, wherein a mol % y_{c} of the PHA is 70 to 95 mol % of the PHA/PLA copolymer.
